(19) United States
(12) Patent Application Publication
(10) Pub. No.: US 2007/0100321 A1
(43) Pub. Date: May 3, 2007

(54) Medical device

(76) Inventors: Leon Rudakov, Belmont, CA (US); Tsui Ying Rachel Hong, Singapore (SG)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/541,254

(22) PCT Filed: Dec. 22, 2004
(86) PCT No.: PCT/SG04/00425

§ 371 Date: Aug. 1, 2006

(51) Int. Cl. A61M 31/00

(52) U.S. Cl. 604/508; 604/60

(57) ABSTRACT

A medical device (10) for insertion into a bodily vessel (6) to treat an aneurysm (5) having an aneurysm neck, the device (10) comprising: a mechanically expandable device (20) expandable from a first position to a second position, said mechanically expandable device (20) is expanded radially outwardly to the second position such that the exterior surface of said mechanically expandable device (20) engages with the inner surface of the vessel (6) so as to maintain a fluid pathway through said vessel (6); a therapeutically effective amount of a chemical compound comprising a biosynthesis accelerator to stimulate cell growth; and a polymer (30, 41, 42) mixed with the chemical compound to manage the release rate of the chemical compound; wherein the mechanically expandable device (20) provides a support for the release of the chemical compound within the aneurysm (5) to stimulate cell growth within the aneurysm (5) and close the aneurysm neck.

### TECHNICAL FIELD

The invention concerns a medical device for insertion into a bodily vessel to treat an aneurysm having an aneurysm neck.

### BACKGROUND OF THE INVENTION

Intracranial aneurysms are currently treated by engaging neurosurgical clipping or using several minimally invasive techniques. For example, interventional neuroradiology uses minimally invasive methods to treat aneurysms. Other methods include: coiling, stenting and coiling; and using gels, glues, or fibrin sealants.

There is a desire to treat aneurysms such that it does not leave any mass (such as solid coils) or foreign body material in a healed aneurysm.

### SUMMARY OF THE INVENTION

In a first preferred aspect, there is provided a medical device for insertion into a bodily vessel to treat an aneurysm having an aneurysm neck, the device comprising:
a mechanically expandable device expandable from a first position to a second position, said mechanically expandable device is expanded radially outwardly to the second position such that the exterior surface of said mechanically expandable device engages with the inner surface of the vessel so as to maintain a fluid pathway through said vessel;
a therapeutically effective amount of a chemical compound comprising a biosynthesis accelerator to stimulate cell growth; and
a polymer mixed with the chemical compound to manage the release rate of the chemical compound;
wherein the mechanically expandable device provides a support for the release of the chemical compound within the aneurysm to stimulate cell growth within the aneurysm and close the aneurysm neck.

The accelerator may be a threo-1-phenyl-2-decanoylamino-3-morpholino-1-propanol compound. Specifically, the accelerator may be L-threo-1-phenyl-2-decanoylamino-3-morpholino-1-propanol (L-PDMP) and therapeutically acceptable salts thereof.

Synthetic ceramide analog, L-PDMP, may stimulate the biosynthesis of glycosphingolipids (GSL) such as Lactosylceramide (LacCer) and glucosylceramide (GIcCer), which in turn stimulates cell growth.

The polymer may be biocompatible, biodegradable, hydrophilic, and has a high degree of swelling.

The polymer may be in a solid or highly viscous form, or is highly elastic.

The polymer may comprise a hydrophilic shell and a hydrophobic core or solely consists of a hydrophilic composition.

The polymer may be selected from the group consisting of: synthetic biodegradable polymers such as Poly (glycolic acid) (PGA), Poly (lactic acid) (PLA), Poly (lactic-co-glycolic acid) (PLGA), poly (ecaprolactone), Polyanhydride, poly (orthoesters), polyphosphazane; biodegradable polymers from natural sources such as modified polysaccharides (cellulose, chitin, dextran) and Modified proteins (fibrin, casein); and hydrogels or superabsorbants such as Poly (ethylene oxide) (PEO), Poly (ethylene glycol) PEG, Methylacrylate (MAA), Maleic anhydride (MAH), Polyacrylamide, Poly (hydroxyethyl methacrylate), Poly (N-vinyl pyrrolidone), Poly (vinyl alcohol).

The L-PDMP compound may be coated on 2D or 3D platinum coils.

The mechanically expandable device may comprise a generally tubular structure having an exterior surface defined by a plurality of interconnected struts having interstitial spaces therebetween.

The polymer and chemical compound may be released into the aneurysm by a delivery catheter passing through the mechanically expandable device and between the struts of the mechanically expandable device proximal to the aneurysm.

The polymer and chemical compound may be in the form of micro-spheres, spherical, or cylindrical (with coils).

The delivery catheter may comprise a distal compartment for securing the chemical compound, and a proximal compartment, the distal and proximal compartments being separated by an elastic membrane, wherein pressure applied to the proximal compartment is translated to the distal compartment causing the polymer and chemical compound to be released from the delivery catheter into the aneurysm.

The delivery catheter may further comprise a valve to allow exit of the polymer and chemical compound but prevents blood from entering the delivery catheter.

The polymer and the chemical compound may be in the form of a membrane attached to the outer surface of the mechanically expandable device, such that when the mechanically expandable device is expanded, the membrane faces the aneurysm and the chemical compound is released towards the aneurysm.

The membrane may be a single layer or comprises multiple layers.

The membrane may be biodegradable.

The polymer may be solid or porous.

The polymer may be amorphous or semi-crystalline.

The device may further comprise radiopaque markers incorporated in the polymer to improve the visibility of the polymer and chemical compound during deployment.

The device may further comprise radiopacifers such as barium sulphate, zirconium dioxide or iodine.

The mechanically expandable device may be biodegradable.

The mechanically expandable device and polymer may biodegrade at different rates.

In a second aspect, there is provided a method for treating an aneurysm having an aneurysm neck, the method comprising:
positioning a mechanically expandable device into a bodily vessel proximate to the aneurysm neck;
releasing a therapeutically effective amount of a chemical compound comprising a biosynthesis accelerator to stimulate cell growth within the aneurysm;
wherein the mechanically expandable device provides a support for the release of the chemical compound within the aneurysm to stimulate cell growth within the aneurysm and close the aneurysm neck.

The method may further comprise passing a delivery catheter through the mechanically expandable device and between the struts of the mechanically expandable device proximal to the aneurysm, to deliver the chemical compound.

The method may further comprise mechanically pushing the chemical compound from the delivery catheter and into the aneurysm.

The method may further comprise applying pressure in a proximal compartment of the delivery catheter to cause the chemical compound to be pushed out of a distal compartment of the delivery catheter and into the aneurysm.

### BRIEF DESCRIPTION OF THE DRAWINGS

An example of the invention will now be described with reference to the accompanying drawings, in which:

FIG. 1 is an illustration of the molecular structure of Poly (glycolic acid);

FIG. 2 is an illustration of the molecular structure of Poly (lactic acid);

FIG. 3 is an illustration of the molecular structure of Poly (lactic-co-glycolic acid);

FIG. 4 is a diagrammatic view of a delivery catheter delivering the polymer and L-PDMP compound;

FIG. 5 is a diagrammatic view of the polymer in two forms;

FIG. 6 is a diagrammatic view of the polymer in membrane form;

FIG. 7 is an illustration of the molecular structure of L-PDMP;

FIG. 8 is a diagrammatic view of a stent positioned across an aneurysm;

FIG. 9 is a diagrammatic view of the delivery catheter delivering the polymer and L-PDMP compound into the aneurysm;

FIG. 10 is a diagrammatic view of the polymer and L-PDMP compound filling the aneurysm and embolising;

FIG. 11 is a diagrammatic view of a membrane attached to the stent for releasing the L-PDMP compound into the aneurysm;

FIG. 12 is a diagrammatic view of the L-PDMP compound degrading and the aneurysm healing; and

FIG. 13 is a diagrammatic view of the membrane biodegrading and the aneurysm healing.

### DETAILED DESCRIPTION OF THE DRAWINGS

Referring to the drawings, the medical device generally comprises three components: a stent 20, a polymer 30, 41, 42 and L-threo-1-Phenyl-2-Decanoylamino-3-Morpholino-1-Propanol (L-PDMP) compound. A first embodiment of the medical device comprises the stent 20 and a biodegradable, hydrophilic polymer 30 mixed with the L-PDMP compound. A second embodiment of the medical device comprises the stent 20 with a biodegradable membrane 41, 42 with at least one layer of the hydrophilic polymer 30.

The stent 20 may be made of the following materials utilizing different deployment mechanisms:
Balloon expandable stent made from: stainless steel, PtW alloy, or Ti;
Self-expandable stent made from NiTi; or
Biodegradable stent.

If the stent 20 is deployed by balloon expansion, it is made from stainless steel, platinum tungsten alloy or titanium. If the stent 20 is deployed by self expansion, it is made from Nitinol.

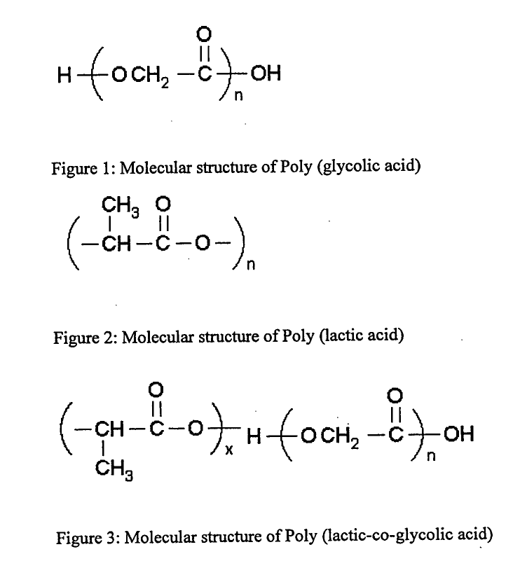

Suitable biodegradable materials for the stent 20 include:
Poly (glycolic acid) (PGA) as shown in FIG. 1;
Poly (lactic acid) (PLA) as shown in FIG. 2;
Poly (lactic-co-glycolic acid) (PLGA) as shown in FIG. 3;
Poly (ecaprolactone) (PCL);
Polyanhydride (PA); or
Poly (orthoesters) (POE).

If the stent 20 is made from a biodegradable material, foreign material in the vessel 6 is reduced or eliminated after the aneurysm 5 is healed. The stent 20 also biodegrades while the aneurysm 5 is healing.

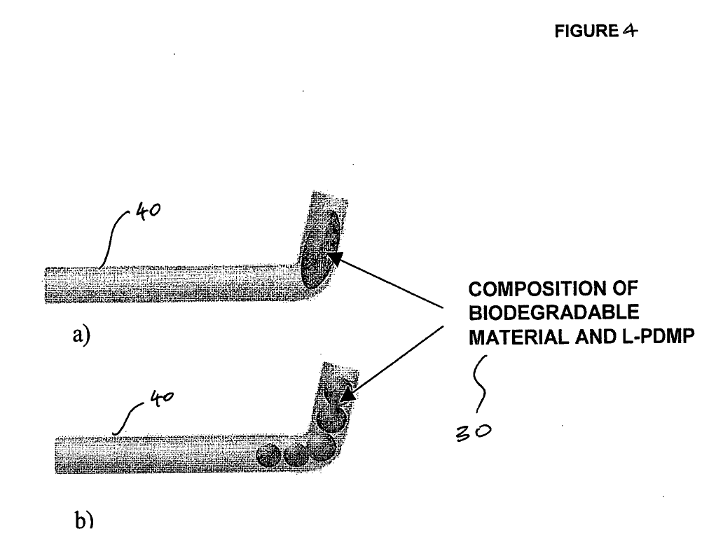

Referring to FIGS. 4, 5 and 6, the polymer 30, 41, 42 is a medium for the attaching the L-PDMP compound. The polymer 30, 41, 42 manages the release rate of the L-PDMP compound and also provides a scaffold for cell growth. The shape of the polymer 30, 41, 42 may include: micro-spheres 30, spherical 30, cylindrical (with coils), or be in the form of a thin membrane 41, 42.

The polymer 30 is biocompatible, biodegradable, hydrophilic, has a high degree of swelling. The polymer 30 has a fast swelling rate (from instantaneous to approximately 5 to 6 minutes). The polymer 30 may be in a solid or highly viscous form, or is highly elastic.

The polymer 30 is based on any one of the following materials:
Synthetic biodegradable polymer such as Poly (glycolic acid) (PGA), Poly (lactic acid) (PLA), Poly (lactic-co-glycolic acid) (PLGA), poly (ecaprolactone), Polyanhydride, poly (orthoesters), polyphosphazane;
Biodegradable polymers from natural sources such as modified polysaccharides (cellulose, chitin, dextran) and Modified proteins (fibrin, casein); and
Hydrogels or superabsorbants such as Poly (ethylene oxide) (PEO), Poly (ethylene glycol) PEG, Methylacrylate (MAA), Maleic anhydride (MAH), Polyacrylamide, Poly (hydroxyethyl methacrylate), Poly (N-vinyl pyrrolidone), Poly (vinyl alcohol).

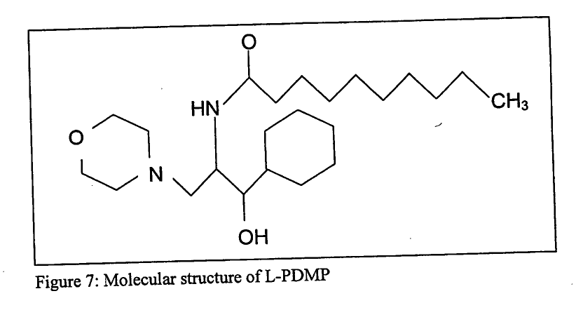

Referring to FIG. 7, L-PDMP is a chemical compound which promotes a glycolipid biosynthesis-accelerating effect. This is described in U.S. Pat. No. 5,041,441 and Japanese Patent 254623/1989. L-PDMP or its derivatives are used to enhance healing and facilitate closing of the aneurysm 5. L-PDMP is used with other types of enzyme GaIT-2 enhancing compounds (including L-PDMP and its derivatives) for the purpose of cell proliferation, including targeting cells such as endothelial, smooth muscle and other types of cells that are available in the intracranial vascular system. Cell proliferation embolizes and effectively obstructs blood circulation to the aneurysm 5. Also, the aneurysm 5 is naturally healed because the aneurysm 5 is deprived of blood circulation and nutrient supply.

The L-PDMP compound is locally released within the aneurysm 5. The release profile of the L-PDMP compound has an initial burst release within the first few hours, to activate biosynthesis and form an outer sphere of emboli, thus enhancing the process of closing the aneurysm neck 5 with a biological cell based substrate. This is followed by a steady state release lasting for 1 to 2 weeks. The L-PDMP compound is designed to activate biosynthesis after it is released. The L-PDMP compound stimulates the biosynthesis of glycosphingolipids (GSL), specifically Lactosylceramide (LacCer) and glucosylceramide (GIcCer). GSLs exist as constitutional component of cell surface membranes and are closely related to a cellular function. GIcCer is precursors for other complex GSLs and are involved in proliferation of cells. LacCer is present in vascular cells such as smooth muscle cells, endothelial cells, macrophages, neutrophils, platelets and monocytes, all of which are involved in the natural healing process. It also serves as a lipid second messenger that orchestrates a signal transduction pathway, leading to cell proliferation.

The acceleration of GSL biosynthesis leads to the following cellular response:
fibroblast and endothelial cell growth;
promotion of collagen formation and smooth muscle cell proliferation; and
occlusion of the aneurysm and neointima coverage of the aneurysm neck. The aneurysm is removed from normal blood circulation.

The healing process begins when the aneurysm neck 5 is filled by the proliferation of cells activated by the L-PDMP compound. The membrane 30, 41, 42 and stent 20 biodegrade over time.

### EXAMPLE 1

In the first embodiment, the medical device includes a stent 20 with a biodegradable hydrophilic viscous composition 30, that is, a highly viscous solution of biodegradable, hydrophilic material mixed with the L-PDMP compound. In a specific example, the L-PDMP compound is coated on 2D or 3D platinum coils. Alternatively, one coil is used in parallel with gel spheres used as markers.

The stent 20 assists with the delivery of the L-PDMP compound to a selected aneurysm site 5 by supporting or scaffolding the vessel 6 and protecting and securing the L-PDMP composition introduced into the aneurysm 5. A delivery catheter 40 is provided to deploy the L-PDMP compound in a controlled manner to treat the aneurysm 5. After the stent 20 is positioned at a selected aneurysm site 5, the L-PDMP compound is deployed using the delivery catheter 40 to create an embolization environment at the aneurysm site 5. This eventually causes the aneurysm neck 5 to close as a result of the biological reaction caused by L-PDMP compound and subsequent biological activity.

The polymer 30 is delivered as a single particle or as connected smaller particles. The microstructure of the polymer 30 may be solid or porous (micropores (10-100 nm), macropores (100 nm-10 μm) or superpores (˜100 μm). The polymer 30 is either amorphous or semi-crystalline. If radiopaque markers are used, platinum coils are incorporated in the polymer 41, 42. Radiopacifers are added to the polymer 41, 42 such as barium sulphate (BaSO₄), zirconium dioxide (ZrO₂) and iodine.

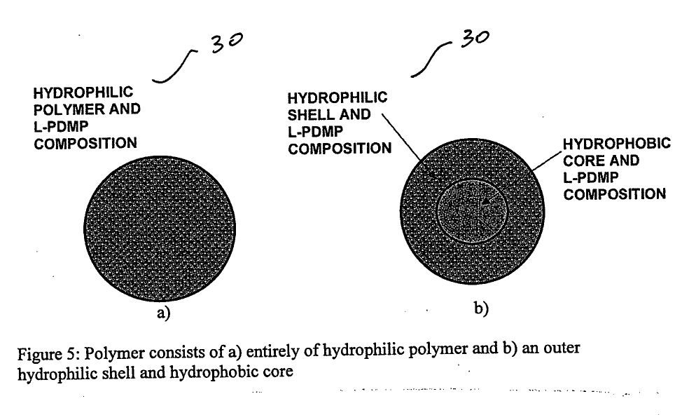

Referring to FIG. 5a, the particle(s) 30 facilitate the rate and degree of swelling as well as the rate of degradation. These particles 30 consist entirely of a hydrophilic polymer, for fast release and degradation. Alternatively, referring to FIG. 5b, the particle(s) 30 consists of an outer shell of a hydrophilic polymer with a core made of hydrophobic polymer, such as polyanhydride, poly (ortho esters) or poly (L-lactic acid), for greater sustained release and extend degradation time if needed.

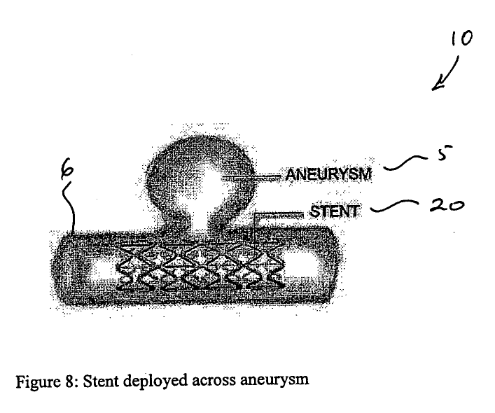

Referring to FIG. 8, the stent 20 is deployed and expanded against the aneurysm neck 5 to create a scaffold or support. The polymer 30 and L-PDMP compound is secured in a distal compartment at the distal tip of the delivery catheter 40. Next, the delivery catheter 40 with the hydrophilic substrate is introduced to the aneurysm 5. The hydrophilic substrate is a mixture of hydrophilic viscous biodegradable material with L-PDMP compound.

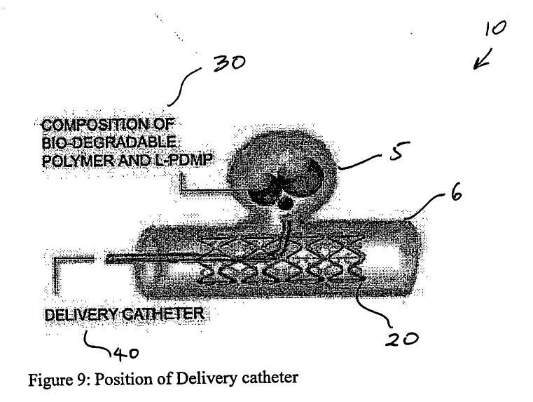

Referring to FIG. 9, the distal tip of the delivery catheter 40 is introduced to the aneurysm neck 5 between the stent struts. When the distal tip is positioned in or near the aneurysm neck 5, the polymer 30 and L-PDMP compound is released from the distal compartment by mechanically pushing the L-PDMP compound with a core wire in the inner lumen of the delivery catheter 40. The tip of the delivery catheter 40 has a valve to allow the L-PDMP compound to exit but prevents blood from entering to reduce premature swelling of the polymer 30 and activation of the L-PDMP. The L-PDMP compound is pushed out of the inner lumen of the delivery catheter 40 by a core wire. The core wire functions similarly to a piston in a hydraulic cylinder.

Another way to deploy the L-PDMP compound is to modify the delivery catheter 40 by providing an inner lumen proximal/mid-shaft compartment and distal compartment within the delivery catheter 40. The L-PDMP compound is secured within the distal compartment. The proximal and distal compartments of the delivery catheter 40 are separated by a super elastic membrane. When pressure is applied to the proximal compartment, the membrane transfers the pressure from proximal compartment to the distal compartment and thus pushes the L-PDMP compound out of the delivery catheter 40 and into the aneurysm 5.

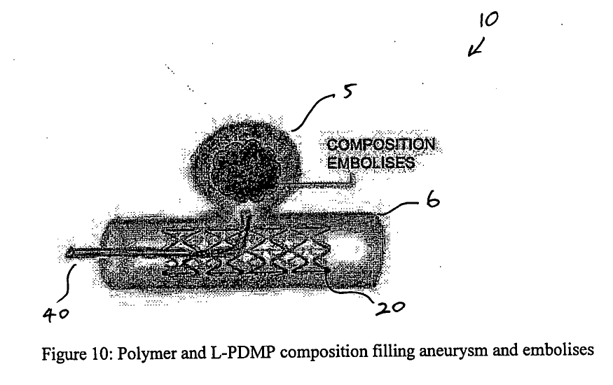

Referring to FIG. 10, upon release, the polymer 30 and L-PDMP compound immediately absorbs the blood within the aneurysm 5 and swells to a size larger than the stent struts, at a fixed rate. The inner space of the aneurysm 5 is filled up after deployment is completed and the L-PDMP compound is released and activated. A biological cell based substrate is formed and swells and expands. It grows in size very quickly size, larger than the distance between stent struts. At this point, the stent struts prevent the substrate from returning towards the vessel. After the substrate occupies the aneurysm dome 5, it starts releasing the L-compound and activating the cell proliferation and embolization process. The L-PDMP compound is designed to be active only during its release and facilitates the embolization process as long as it needed. The L-PDMP compound ceases activity after its release is seized. After the aneurysm dome 5 is filled by newly developed emboli, blood supply into the aneurysm 5 is reduced and eventually stopped. The biodegradable material gradually biodegrades leaving the healing site with a natural vessel wall.

### EXAMPLE 2

In the second embodiment, the medical device includes a stent 20 with a biodegradable membrane 41, 42 made from biodegradable material mixed with the L-PDMP compound. The stent 20 is deployed at the aneurysm site 5 against its neck. The membrane 41, 42 obstructs blood circulation through the aneurysm neck to the aneurysm 5. The L-PDMP compound is encased in layers of the membrane 42. The L-PDMP compound starts to release and activate cell proliferation towards the aneurysm neck and dome 5.

The membrane 41, 42 is made from a mixture of the biodegradable polymer and L-PDMP compound. The direction that the L-PDMP compound is released is controlled and directed outwards towards the vessel wall and aneurysm neck.

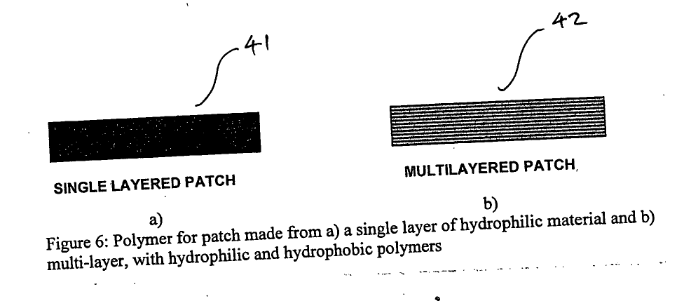

Referring to FIG. 6a and 6b, if the polymer is in the form of a membrane 41, 42 to cover the aneurysm 5, the polymer is a single layer of biodegradable polymer 41 or is multi-layered 42; consisting of both biodegradable materials. The microstructure of the polymer 41, 42 may be solid or porous (micropores (10-100 nm), macropores (100 nm-10 μm) or superpores (˜100 μm). The polymer 41, 42 is either amorphous or semi-crystalline. If radiopaque markers are used, platinum coils are incorporated in the polymer 41, 42. Radiopacifers are added to the polymer 41, 42 such as barium sulphate (BaSO₄), zirconium dioxide (ZrO₂) and iodine.

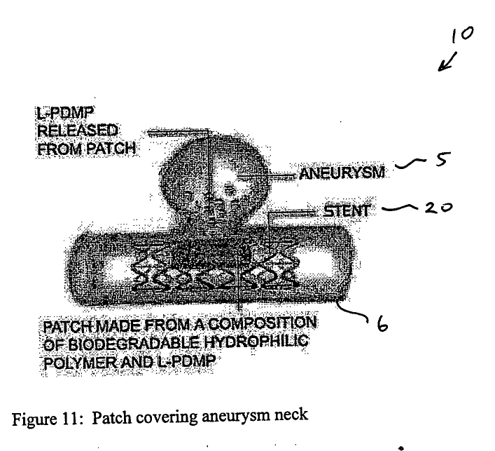

Referring to FIG. 11, a thin film membrane 41 is made of a biodegradable polymer and the L-PDMP compound. The membrane 41 is attached to stent struts. Alternatively, a non-biodegradable polymer can be used. When the stent 20 is deployed, the membrane 41 obstructs blood circulation through the neck of the aneurysm 5. The L-PDMP compound is activated and released towards the aneurysm neck and dome 5.

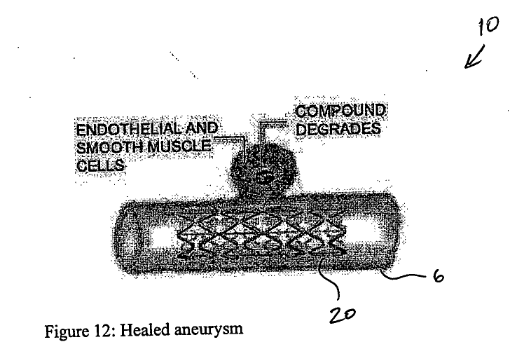

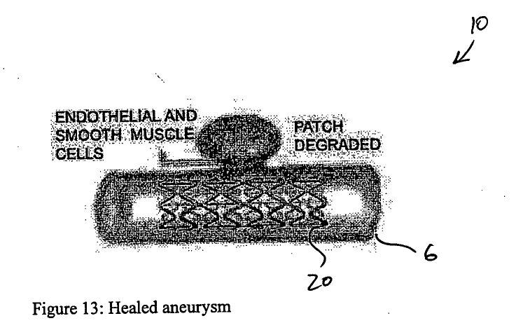

Referring to FIGS. 12 and 13, the polymer 30, 41, 42 slowly degrades after deployment. The degradation/release time varies from 10 to 14 days to 1 to 2 months. The degradation is controllable by mechanisms and structures described. This enables the aneurysm to 5 heal completely, and leaves a natural vessel wall 6.

The medical device is suitable for different aneurysm sizes, including small aneurysms (<15 mm), large aneurysms (15-25 mm), giant aneurysms (25-50 mm) as well as different aneurysm types such as Berry aneurysm or wide neck aneurysm (neck>4 mm and/or dome-to-neck ratio<2).

It will be appreciated by persons skilled in the art that numerous variations and/or modifications may be made to the invention as shown in the specific embodiments without departing from the scope or spirit of the invention as broadly described.

The present embodiments are, therefore, to be considered in all respects illustrative and not restrictive.

1. A medical device for insertion into a bodily vessel to treat an aneurysm having an aneurysm neck, the device comprising:
a mechanically expandable device expandable from a first position to a second position, said mechanically expandable device is expanded radially outwardly to the second position such that the exterior surface of said mechanically expandable device engages with the inner surface of the vessel so as to maintain a fluid pathway through said vessel;
a therapeutically effective amount of a chemical compound comprising a biosynthesis accelerator to stimulate cell growth; and
a polymer mixed with the chemical compound to manage the release rate of the chemical compound;
wherein the mechanically expandable device provides a support for the release of the chemical compound within the aneurysm to stimulate cell growth within the aneurysm and close the aneurysm neck.

2. The device according to claim 1, wherein the accelerator is a threo-1-phenyl-2-decanoylamino-3-morpholino-1-propanol compound.

3. The device according to claim 2, wherein the accelerator is L-threo-1-phenyl-2-decanoylamino-3-morpholino-1-propanol (L-PDMP) and therapeutically acceptable salts thereof.

4. The device according to claim 3, wherein the L-PDMP compound stimulates the biosynthesis of glycosphingolipids (GSL).

5. The device according to claim 4, wherein the L-PDMP compound stimulates the biosynthesis of Lactosylceramide (LacCer) and glucosylceramide (GlcCer).

6. The device according to claim 1, wherein the polymer is biocompatible, biodegradable, hydrophilic, and has a high degree of swelling.

7. The device according to claim 6, wherein the polymer is in a solid or highly viscous form, or is highly elastic.

8. The device according to claim 1, wherein the polymer comprises a hydrophilic shell and a hydrophobic core or solely consists of a hydrophilic composition.

9. The device according to claim 1, wherein the polymer is selected from the group consisting of: synthetic biodegradable polymers such as Poly (glycolic acid) (PGA), Poly (lactic acid) (PLA), Poly (lactic-co-glycolic acid) (PLGA), poly (ecaprolactone), Polyanhydride, poly (orthoesters), polyphosphazane; biodegradable polymers from natural sources such as modified polysaccharides (cellulose, chitin, dextran) and Modified proteins (fibrin, casein); and hydrogels or superabsorbants such as Poly (ethylene oxide) (PEO), Poly (ethylene glycol) PEG, Methylacrylate (MAA), Maleic anhydride (MAH), Polyacrylamide, Poly (hydroxyethyl methacrylate), Poly (N-vinyl pyrrolidone), Poly (vinyl alcohol).

10. The device according to claim 3, wherein the L-PDMP compound is coated on 2D or 3D platinum coils.

11. The device according to claim 1, wherein the mechanically expandable device comprises a generally tubular structure having an exterior surface defined by a plurality of interconnected struts having interstitial spaces therebetween.

12. The device according to claim 11, wherein the polymer and the chemical compound are released into the aneurysm by a delivery catheter passing through the mechanically expandable device and between the struts of the mechanically expandable device proximal to the aneurysm.

13. The device according to claim 12, wherein the polymer and the chemical compound are in the form of micro-spheres, spherical, or cylindrical (with coils).

14. The device according to claim 12, wherein the delivery catheter comprises a distal compartment for securing the polymer and the chemical compound, and a proximal compartment, the distal and proximal compartments being separated by an elastic membrane, wherein pressure applied to the proximal compartment is translated to the distal compartment causing the polymer and the chemical compound to be released from the delivery catheter into the aneurysm.

15. The device according to claim 14, wherein the delivery catheter further comprises a valve to allow exit of the polymer and the chemical compound but prevents blood from entering the delivery catheter.

16. The device according to claim 1, wherein the polymer and the chemical compound are in the form of a membrane attached to the outer surface of the mechanically expandable device, such that when the mechanically expandable device is expanded, the membrane faces the aneurysm and the chemical compound is released towards the aneurysm.

17. The device according to claim 16, wherein the membrane is a single layer or comprises multiple layers.

18. The device according to claim 16, wherein the membrane is biodegradable.

19. The device according to claim 16, wherein the polymer is solid or porous.

20. The device according to claim 16, wherein the polymer is amorphous or semi-crystalline.

21. The device according to claim 1, further comprising radiopaque markers incorporated in the polymer to improve the visibility of the polymer and chemical compound during deployment.

22. The device according to claim 21, further comprising radiopacifers such as barium sulphate, zirconium dioxide or iodine.

23. The device according to claim 1, wherein the mechanically expandable device is biodegradable.

24. The device according to claim 23, wherein the mechanically expandable device and polymer biodegrade at different rates.

25. A method for treating an aneurysm having an aneurysm neck, the method comprising:
positioning a mechanically expandable device into a bodily vessel proximate to the aneurysm neck;
releasing a therapeutically effective amount of a chemical compound comprising a biosynthesis accelerator to stimulate cell growth within the aneurysm;
wherein the mechanically expandable device provides a support for the release of the chemical compound within the aneurysm to stimulate cell growth within the aneurysm and close the aneurysm neck.

26. The method according to claim 25, further comprising passing a delivery catheter through the mechanically expandable device and between the struts of the mechanically expandable device proximal to the aneurysm, to deliver the chemical compound.

27. The method according to claim 26, further comprising mechanically pushing the chemical compound from the delivery catheter and into the aneurysm.

28. The method according to claim 26, further comprising applying pressure in a proximal compartment of the delivery catheter to cause the chemical compound to be pushed out of a distal compartment of the delivery catheter and into the aneurysm.
